(19) United States
(12) Patent Application Publication
(10) Pub. No.: US 2013/0012840 A1
(43) Pub. Date: Jan. 10, 2013

(54) SYSTEM AND METHOD FOR ACCELERATING THE RELATIVE PROPORTION OF FAT CATABOLISM

(75) Inventor: Ilan Feferberg, Jerusalem (IL)

(73) Assignee: REAL AESTHETICS LTD., Jerusalem (IL)

(21) Appl. No.: 13/636,355

(22) PCT Filed: Mar. 23, 2011
(86) PCT No.: PCT/IL2011/000271

§ 371 Date: Sep. 21, 2012

Related U.S. Application Data
(60) Provisional application No. 61/316,420, filed on Mar. 23, 2010.

(51) Int. Cl. A61N 7/00; A61N 1/00

(52) U.S. Cl. 601/2

(57) ABSTRACT

System and method for reducing stored body fats in a treatment region on a body. The system includes an ultrasound apparatus, for transmitting ultrasound waves at the treatment region. The ultrasound waves facilitate the release of triglycerides, fat molecules and toxins from stored fat within the treatment region, achieving the reduction of stored body fats by accelerating the relative proportion of fat catabolism in the treatment region. The treated person may be undergoing a physical exercise substantially concurrently to the ultrasound transmission. Electrical stimulation, such as interferential stimulation, may be applied to the treatment region substantially concurrently to the ultrasound transmission. Pressure may be exerted onto the treatment region substantially concurrently to the ultrasound transmission, such as by kneading a portion of the ultrasound apparatus against the treatment region, or by massaging the treatment region, manually or mechanically with a massaging device.

### FIELD OF THE DISCLOSED TECHNIQUE

The disclosed technique relates to body fat reduction systems and methods.

### BACKGROUND OF THE DISCLOSED TECHNIQUE

It is estimated that approximately 65 percent of American adults are considered overweight and approximately 31 percent are considered obese and at risk for chronic diseases resulting from excess fat. In addition to the health risks, excess body fat is generally unappealing, detrimentally affects one's self-image, and involves various physically hardships. Many individuals with excess body fat attempt many different body fat reduction methods with limited success, for reasons which include: genetics, improper eating habits, insufficient intake of water, smoking, lifestyle factors, self discipline, tension and stress, medication, hormonal balances, metabolism, and other reasons. In many cases, individuals are able to achieve some body fat reduction, but cannot seem to reduce body fat in specific body parts or regions of their bodies. Common methods of attempted fat reduction involve reduced caloric intake and exercise.

There are three main categories of organic matter from which energy is obtained in a living organism: carbohydrates, fats (lipids), and proteins. The process of catabolism is the metabolic stage in which the organic molecules are broken down into smaller units to release energy through cellular respiration. When undergoing physical exercise, the body obtains energy predominantly from the carbohydrates of recently consumed food, which are the most readily available source of energy. Initially, the body breaks down (i.e., catabolises) mainly carbohydrates, along with a smaller percentage of fats and proteins. Once a significant proportion of the stored carbohydrates have been depleted, the body begins to catabolise stored body fats at a higher proportion relative to the fat catabolism during the beginning of the exercise session. Therefore, an individual with the goal of reducing stored body fat must exercise for a sufficient duration for the body to complete the initial metabolic stage, which involves a significantly higher relative proportion of carbohydrate catabolism, and enters the subsequent metabolic stage, during which stored fat is catabolised in a relatively higher proportion than during the initial metabolic stage. However, many people do not manage to reach and remain in the higher proportion of fat catabolism exercise phase for long, and thus do not succeed in reducing stored body fat. This is especially the case if they maintain a high calorie diet relative to the amount of exercise that they perform.

Reference is now made to FIG. 1, which is a schematic illustration of a region of a body, generally referenced 100, for which fat reduction is desired. Body region 100 includes a skin layer 102, a subcutaneous fat layer 104, and a muscle layer 112. Skin layer 102, provides the outer protective layer of the body. Subcutaneous fat layer 104 includes fat cells 106 and connective tissues 108. A proper supply of nutrients and oxygen in the blood supply keeps the fatty tissue well nourished, and a good drainage system by the veins and the lymph channels constantly removes waste. Normal fatty tissue is smooth, well nourished, and free from toxins and excess fluid, but if the blood supply or drainage system becomes disrupted or constricted, then a gradual build-up of toxins and fluids within the fatty tissue may occur. As a result, the body does not succeed in breaking down and removing the accumulated fat.

Fat reduction methods known in the art attempt to help the body break down and eliminate excess stored fats (along with the buildup of fluids and toxins in the fatty tissue). The difficulties of fat reduction are at least partially attributed to the fact that, during physical exercise, catabolism of fat molecules in significant proportions in the body is first preceded by the catabolism of predominantly the available carbohydrates. Additionally, people store fat in different “problematic regions” of their bodies, and even when the body is harvesting energy from stored fat within the body, it may not necessarily be from the stored fat located in the regions where fat reduction is specifically desired.

### SUMMARY OF THE DISCLOSED TECHNIQUE

In accordance with one aspect of the disclosed technique, there is thus provided a system for reducing stored body fats in a treatment region on the body of a treated person. The system includes an ultrasound apparatus, for transmitting ultrasound waves at the treatment region. The ultrasound waves facilitate the release of triglycerides, fat molecules and toxins from stored fat within the treatment region, achieving the reduction of stored body fats by accelerating the relative proportion of fat catabolism in the treatment region. The frequency of the transmitted ultrasound waves is preferably between 1-3 MHz. The frequency of the transmitted ultrasound waves may be altered according to a frequency variation pattern over the course of a treatment session. The intensity of the transmitted ultrasound waves is preferably between 1.5-2.1 W/cm². The treated person may be undergoing a physical exercise substantially concurrently to the transmission of the ultrasound waves. The system may further include an exercise apparatus used by the treated person to perform the physical exercise. The physical exercise is preferably performed at an intensity level of approximately 60% of maximum heart rate. The ultrasound apparatus may be worn by the treated person on the treatment region while performing the physical exercise. The system may further include a muscle stimulation apparatus, for applying electrical stimulation to the treatment region substantially concurrently to the transmission of the ultrasound waves, to stimulate intermittent contractions of the muscles at the treatment region. The electrical stimulation is preferably interferential stimulation, such as premodulated, biphasic, interferential isoplanar (4 poles), interferential vectorial (4 poles), or medium frequency interferential stimulation. The current intensity of the electrical stimulation is preferably between 5-90 mA. The frequency of the electrical stimulation is preferably between 5-150 Hz. The frequency of the electrical stimulation may be altered according to a frequency variation pattern. Pressure may be exerted onto the treatment region substantially concurrently to the transmission of the ultrasound waves at the treatment region. The pressure may be exerted by manually kneading a portion of the ultrasound apparatus against the treatment region. The ultrasound apparatus may further massage the treatment region substantially concurrently to the transmission of the ultrasound waves at the treatment region. The system may further include a massaging device, for massaging the treatment region substantially concurrently to the transmission of the ultrasound waves at the treatment region. The treatment region may be manually massaged substantially concurrently to the transmission of the ultrasound waves at the treatment region. A gel may be applied onto the treatment region prior to the transmission of the ultrasound waves at the treatment region. The system may further include a measurement apparatus, for measuring physical parameters of the treated person, substantially concurrently to the treatment. The system may further include a camera, for recording the treatment.

In accordance with another aspect of the disclosed technique, there is further provided a method for reducing stored body fats in a treatment region on the body of a treated person. The method includes the procedure of transmitting ultrasound waves at the treatment region. The ultrasound waves facilitate the release of triglycerides, fat molecules and toxins from stored fat within the treatment region, achieving the reduction of stored body fats by accelerating the relative proportion of fat catabolism in the treatment region. The frequency of the transmitted ultrasound waves is preferably between 1-3 MHz. The frequency of the transmitted ultrasound waves may be altered according to a frequency variation pattern over the course of a treatment session. The intensity of the transmitted ultrasound waves is preferably between 1.5-2.1 W/cm². The treated person may be undergoing a physical exercise substantially concurrently to the transmission of the ultrasound waves. The physical exercise is preferably performed at an intensity level of approximately 60% of maximum heart rate. The method may further include the procedure of applying electrical stimulation to the treatment region substantially concurrently to the transmission of the ultrasound waves, to stimulate intermittent contractions of the muscles at the treatment region. The electrical stimulation is preferably interferential stimulation, such as premodulated, biphasic, interferential isoplanar (4 poles), interferential vectorial (4 poles), or medium frequency interferential stimulation. The current intensity of the electrical stimulation is preferably between 5-90 mA. The frequency of the electrical stimulation is preferably between 5-150 Hz. The frequency of the electrical stimulation may be altered according to a frequency variation pattern. The method may further include the procedure of exerting pressure onto the treatment region substantially concurrently to the transmission of the ultrasound waves at the treatment region. The pressure may be exerted by manually kneading a portion of the ultrasound apparatus against the treatment region. The method may further include the procedure of massaging the treatment region substantially concurrently to the transmission of the ultrasound waves at the treatment region. The massaging may be applied using the ultrasound apparatus. The massaging may be applied using a massaging device. The massaging may be applied manually. A gel may be applied onto the treatment region prior to the transmission of the ultrasound waves at the treatment region. The method may further include the procedures of acquiring initial measurements of the treated person prior to the procedure of transmitting ultrasound waves, and acquiring final measurements of the treated person after the procedure of transmitting ultrasound waves. The method may further include the procedure of recording the treatment.

### BRIEF DESCRIPTION OF THE DRAWINGS

The disclosed technique will be understood and appreciated more fully from the following detailed description taken in conjunction with the drawings in which:

FIG. 1 is a schematic illustration of a region of a body for which fat reduction is desired;

FIG. 2 is a block diagram of a system for accelerating the relative proportion of stored fat catabolism, constructed and operative in accordance with an embodiment of the disclosed technique;

FIG. 3 is a schematic illustration of the system of FIG. 2 treating a body region of a person, in accordance with an embodiment of the disclosed technique;

FIG. 4 is a schematic illustration of physical and biological processes that occur during the application of the disclosed technique;

FIG. 5A is a graph that depicts a first exemplary variation of ultrasound wave frequency as a function of time, in accordance with an embodiment of the disclosed technique;

FIG. 5B is a graph that depicts a second exemplary variation of ultrasound wave frequency as a function of time, in accordance with an embodiment of the disclosed technique;

FIG. 6, is a schematic illustration of an ultrasound apparatus used during a treatment session while the treated person is exercising, constructed and operative in accordance with another embodiment of the disclosed technique;

FIG. 7 is a flow diagram of a method for accelerating the relative proportion of stored fat catabolism, operative in accordance with an embodiment of the disclosed technique;

FIG. 8 is a table illustrating the relation between (non-protein) R values, KCAL (Kilo-calorie) percentage derived from fats and carbohydrates, respectively, and KCAL per L0₂;

FIG. 9 is a graph illustrating R values as a function of exercise time prior to ultrasound treatment, for a first participant in an experimental study for the disclosed technique;

FIG. 10A is a graph illustrating the R values as a function of exercise time for the first participant in an experimental study for the disclosed technique, after undergoing a placebo treatment;

FIG. 10B is a graph illustrating the R values as a function of exercise time for a second participant in an experimental study for the disclosed technique, after undergoing an ultrasound treatment in accordance with the disclosed technique; and

FIG. 11 is a table summarizing the quantitative metabolic results for all the participants in an experimental study for the disclosed technique.

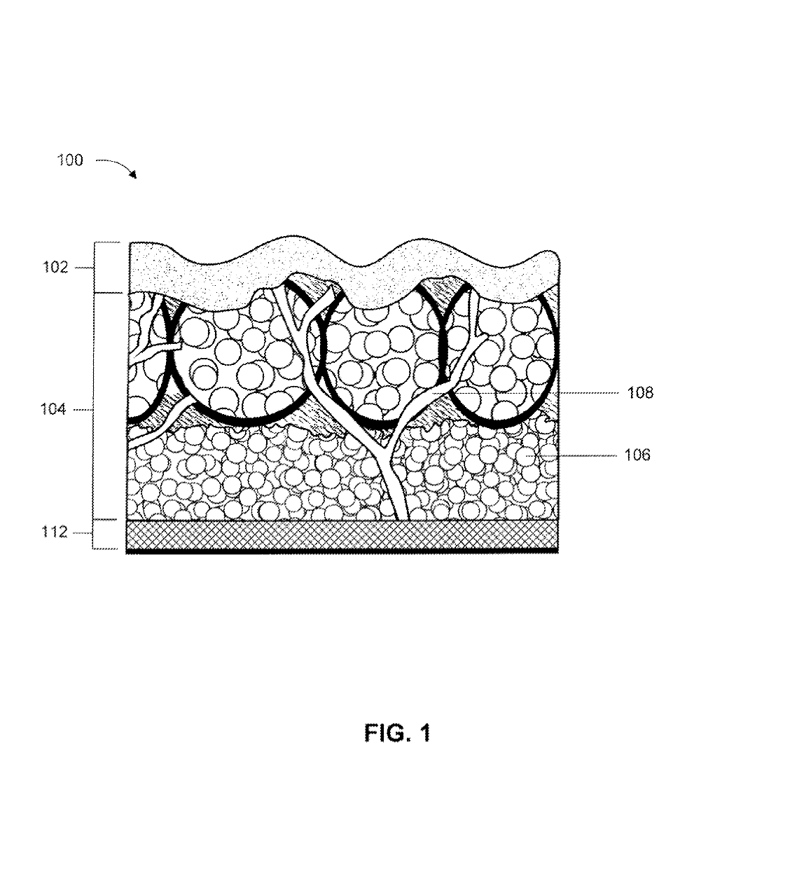

### DETAILED DESCRIPTION OF THE EMBODIMENTS

The disclosed technique facilitates body fat reduction in general and in specific regions of a body by providing a novel system and method that encourages the breaking down of stored fat molecules in the body at a significantly higher proportion than would otherwise have occurred. The system includes an ultrasound apparatus configured to transmit ultrasound waves at specific frequencies and intensities to a particular body region. The transmitted ultrasound waves alter the process by which triglycerides are released from stored body fats, resulting in readily available triglycerides for use in catabolistic processes in the body. The released triglycerides are broken down for energy sooner than they would otherwise have been, enabling quick and effective fat reduction at the treated body region. The disclosed technique may be applied to the treated person substantially concurrently to the person undergoing a physical exercise.

The terms “person”, “treated person”, “individual”, “subject”, “patient”, as well as any grammatical variations thereof, are used interchangeably herein to refer to the person on whom the disclosed technique is being applied. The term “substantially concurrently” as used herein, refers to any period of time immediately before, during, and/or immediately after. Accordingly, by stating that a first procedure is applied “substantially concurrently” to a second procedure, the first procedure may be applied immediately before the second procedure, simultaneous to the second procedure, and/or immediately after the second procedure.

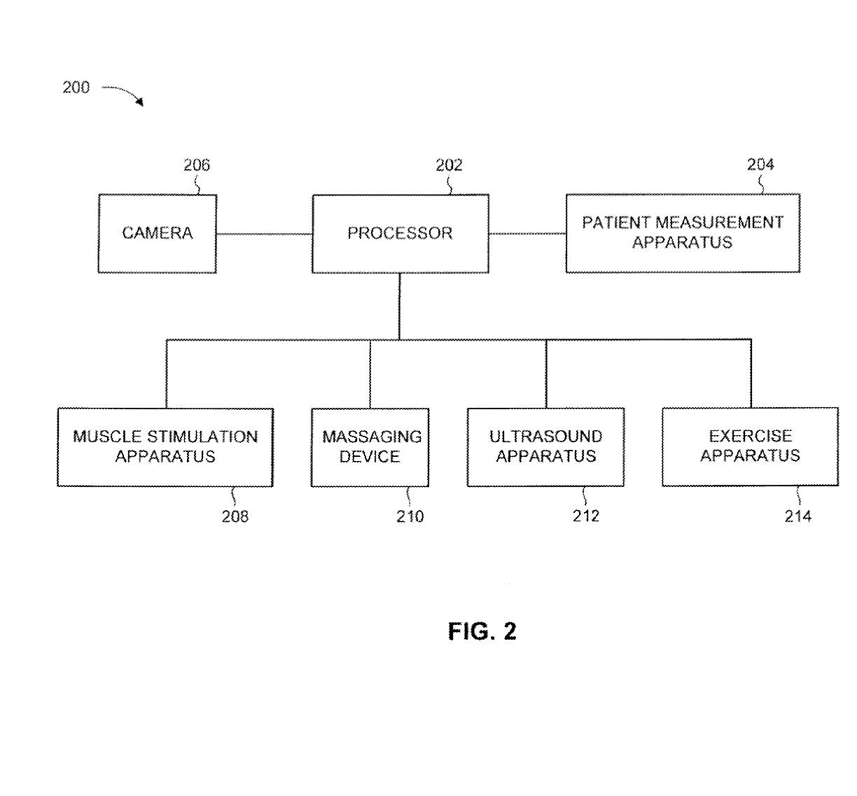

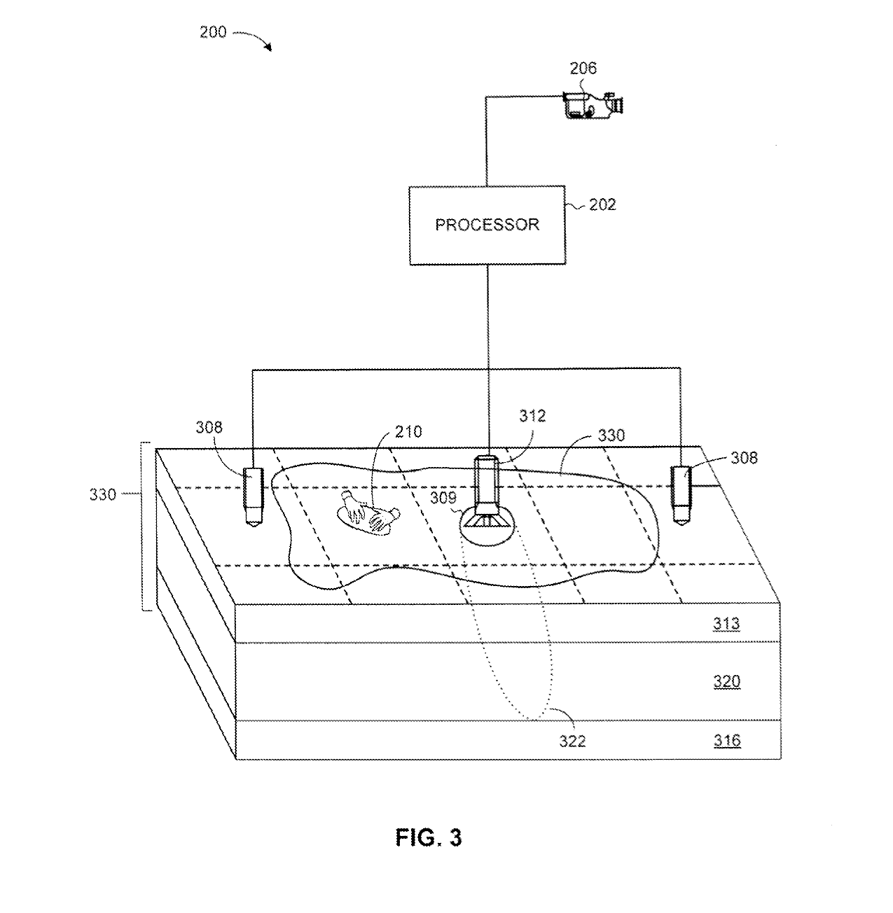

Reference is now made to FIGS. 2 and 3. FIG. 2 is a block diagram of a system for accelerating the relative proportion of stored fat catabolism, generally referenced 200, constructed and operative in accordance with an embodiment of the disclosed technique. FIG. 3 is a schematic illustration of the system of FIG. 2 treating a body region of a person, in accordance with an embodiment of the disclosed technique. System 200 includes a processor 202, a patient measurement apparatus 204, a camera 206, a muscle stimulation apparatus 208, a massaging device 210, an ultrasound apparatus 212, and an exercise apparatus 214 (not shown in FIG. 3). Processor 202 is coupled with and controls patient measurement apparatus 204, camera 206, muscle stimulation apparatus 208, massaging device 210, and ultrasound apparatus 212. Muscle stimulation apparatus 208 includes electrodes 308. Ultrasound apparatus 212 typically includes a signal generator unit (not shown) and an ultrasound transducer 312.

System 200 is applied to a body region 330 on the body of a person, where fat reduction is desired. Body region 330 includes a skin layer 313, a fat layer 320, and a muscle layer 316. Patient measurement apparatus 204 measures physical parameters of the patient, such as: weight, interior or exterior contour of a body part, body fat ratio, and the like. These measurements are taken before, during and/or after the treatment. Processor 202 records the measurements and provides the measurements to a memory (not shown). Camera 206 records the treatment process. The recording can be stored with processor 202 or in a memory (not shown), in order to provide subsequent evidence, to provide additional or alternative measuring means, and/or to assist future treatment improvements.

Electrodes 308 are attached to the patient at body region 330. Muscle stimulation apparatus 208 provides electrical stimulation of muscles 316 of the patient through electrodes 308. Massaging device 210 massages the patient at treatment region 330. Ultrasound apparatus 212 is preferably designed to allow simultaneous kneading of the skin of the patient at the treatment region. System 200 is operative to be applied to body region 330 while the treated person is performing a physical exercise, or alternatively before the body undergoes a physical exercise. Exercise apparatus 214 may be used by the treated person to perform the exercise.

It is appreciated that measurement apparatus 204, camera 206, massaging device 210 and exercise apparatus 214 are optional, and may be absent from alternative embodiments of the disclosed technique. Processor 202 can be eliminated from system 200 if ultrasound apparatus 212 and muscle stimulation apparatus 208 include or are supported by the necessary means to control and manage their operation. Massaging device 210 can be eliminated from system 200 when manual massaging is applied. Muscle stimulation apparatus 208 is preferably only eliminated from system 200 if substantial pressurizing means (such as massaging device 210 or a massaging head of ultrasound apparatus 212) can provide sufficient pressure to stimulate fat molecules, triglycerides and toxins to be extracted from stored fat.

Ultrasound transducer 312 transmits ultrasound waves to treatment region 330. A gel 309 is optionally applied to the patient at the treatment region, to facilitate the application of the ultrasound waves. The ultrasound waves penetrate skin layer 313 until fat layer 320, where the transmitted ultrasound waves proceed to affect these layers. The ultrasound waves are attenuated as they penetrate deeper into the body of the patient, and their intensity and frequency is selected to prevent the undesirable effects that would result if the ultrasound waves reach muscle layer 316, which would be painful and may perhaps harm healthy tissues. A typical cross-section of effective ultrasound penetration for fat layer 320 is represented by perforated lines 322.

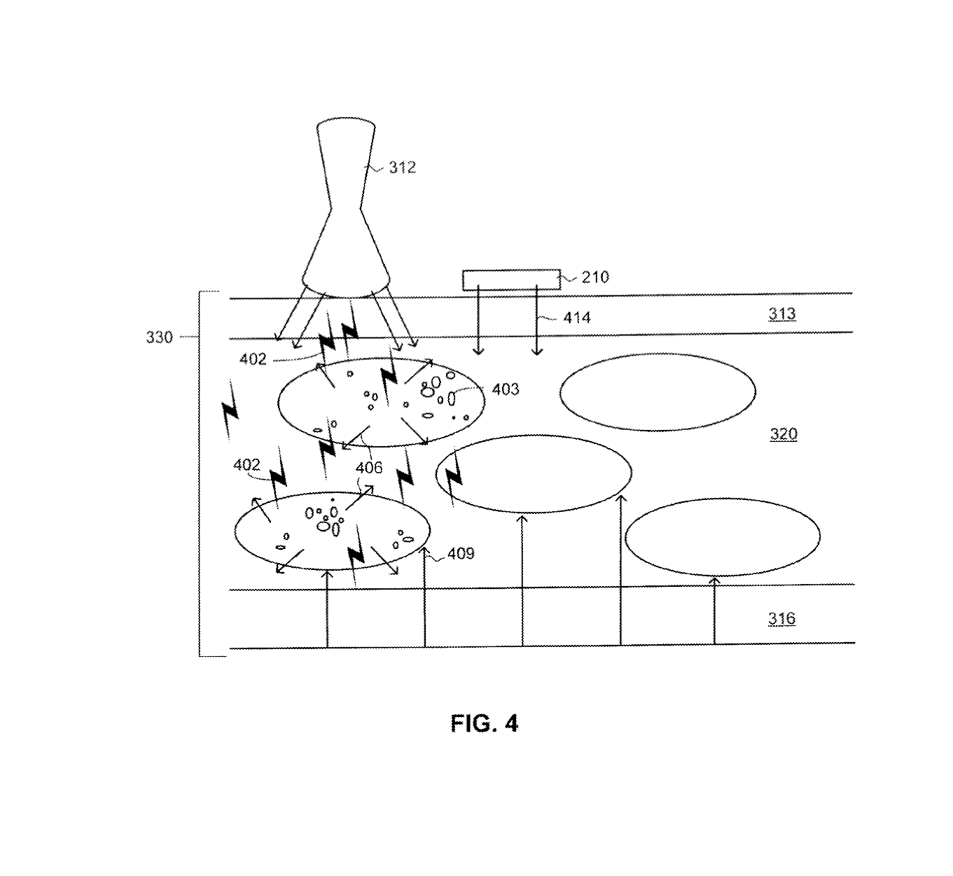

Reference is now made to FIG. 4, which is a schematic illustration of physical and biological processes that occur during the application of the disclosed technique. Ultrasound transducer 312 transmits ultrasound waves 402 at body region 330. Ultrasound waves 402 are very high frequency sound waves that generate heat and create changes in the density and pressure of the medium through which the waves pass. Ultrasound waves 402 are longitudinal waves made up of high pressure regions known as “compression” (where the particles of the medium are compressed together) and low pressure regions known as “rarefaction” (where the particles of the medium are spread apart). When a wave strikes a material, the particles of that material begin to oscillate and gradually generate heat. Thus energy is transferred from the ultrasound wave to the impacted material in the form of thermal energy.

One effect of the ultrasound application is “micro-massage”, which refers to a massage-like process within the molecules of the impacted medium. When ultrasonic waves 402 enter the subcutaneous fat layer, they pass through fat cells 403 and molecules and cause them to vibrate. This vibration generates heat and increases pressure within the fat cells 403, initiating the release of triglycerides from fat cells 403 (as represented by arrows 406) into the circulatory systems of the body, where the triglycerides are catabolized to produce energy, drained out through the circulatory system as waste, or reabsorbed into the stored fat tissue.

It was discovered that the release of triglycerides appears to be most effective at certain frequencies and intensities of ultrasound waves 402. In accordance with the disclosed technique, ultrasound transducer 312 emits ultrasound waves 402 at a frequency between approximately 1 to 4 MHz and at intensities varying from approximately 1 to 3 W/cm². At these ranges, it is conjectured that the micro-massage effect takes place in the stored fat tissue and that deeper healthy tissues, such as muscles, are not harmed. Preferably, the operational frequency of ultrasound transducer 312 is between 1 MHz and 3 MHz, and the operational intensity of ultrasound transducer 312 is between 1.5 and 2.1 W/cm². Lower frequencies should be used when treating fat stored in particularly fat organs to allow deeper penetration. If the patient experiences a burning sensation, then the intensity is reduced, or the ultrasound transducer is moved across the treatment region more quickly in order to shorten the exposure time at a particular region.

The frequency of the ultrasound waves 402 may be varied over the course of the treatment. A change in frequency will allow fat located at different depths in body region 330 to be targeted. Particularly, higher frequencies are used to reach shallower fat layers whereas lower frequencies are used to reach deeper fat layers. When varying the frequency with regard to the depth of fat being targeted, one depth of fat is first treated completely, and subsequently the fat in a different depth is treated.

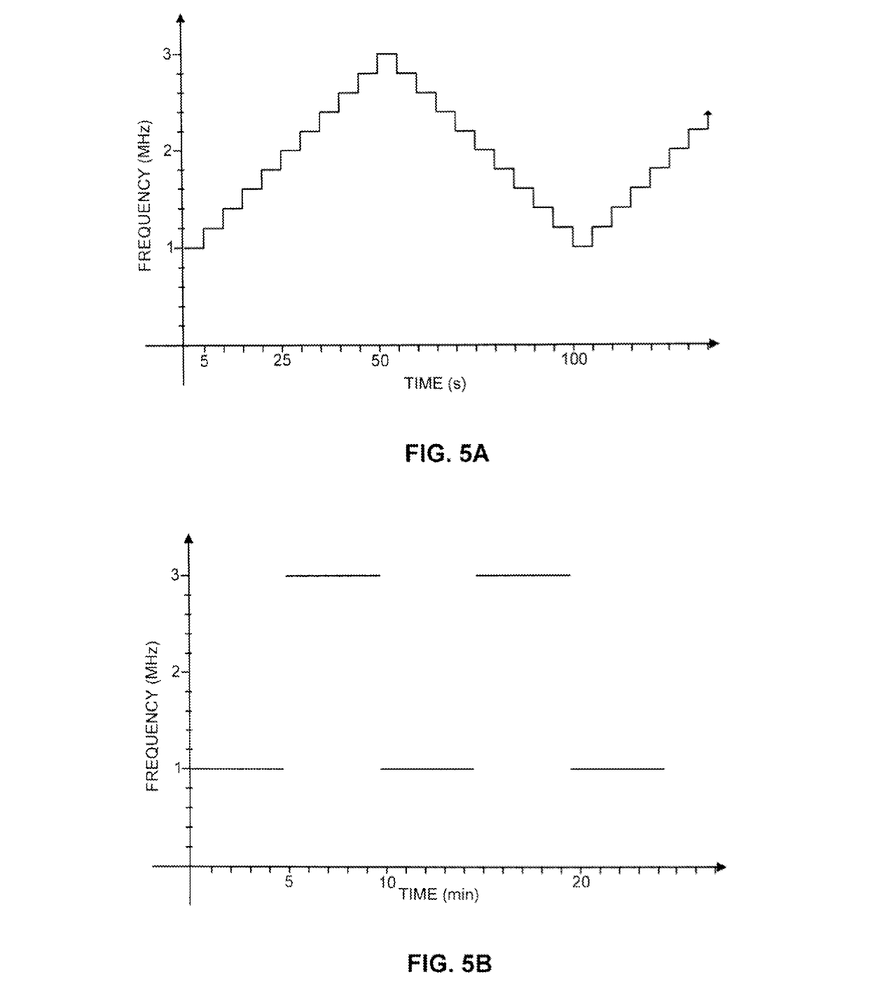

Reference is now made to FIGS. 5A and 5B. FIG. 5A is a graph that depicts a first exemplary variation of ultrasound wave frequency as a function of time, in accordance with an embodiment of the disclosed technique. FIG. 5B is a graph that depicts a second exemplary variation of ultrasound wave frequency as a function of time, in accordance with an embodiment of the disclosed technique. Referring to FIG. 5A, the frequency can be altered over the course of the treatment from 1 MHz to 3 MHz and back again to 1 MHz, cyclically, at steps of 200 KHz lasting 5 seconds. The steps can also last a shorter or longer time period, for example 3 seconds or 10 seconds, and can be larger or smaller in step size, for example 100 KHz or 500 KHz. Referring to FIG. 5B, the frequency can also be altered sharply, in a stepwise manner, between 1 MHz and 3 MHz and back again to 1 MHz, cyclically, where a given frequency is applied for 5 minutes. The duration of a particular frequency applied can also last a shorter or longer time period, for example 3 minutes, 10 minutes, or 20 minutes.

Referring back to FIGS. 3 and 4, during the application of the disclosed technique, a treatment provider slowly and gradually moves ultrasound transducer 312 across the entire body region 330, while preferably executing small circular massage motions with his/her arm and keeping his/her wrist straight. It is noted that the action of the treatment provider may also be automated, such as by a robot or machine. Ultrasound transducer 312 is forcefully applied to treatment region 330 to generate substantial pressure. Ultrasound transducer 312 is preferably designed to allow both the forceful massage action and the penetration of ultrasound waves 402 into body region 330. The changing force of the massaging action, which provides for periodic relief intermissions between the presses, is believed to contribute to the tenability of the organic tissue to an aggressive treatment. Ultrasound transducer 312 can also be tilted in different directions (e.g., left, right, front and back) over the course of the massaging. This is achieved by tilting and moving the wrist in different directions repetitively, for example left-right-left, front-back-front, and left-front-right-back (i.e, circular motion using the wrist as opposed to circular motion using the arm). In this manner, ultrasound waves 402 penetrate deeper into body region 330, as the surface area of the head of ultrasound transducer 312 in contact with the skin is made smaller by the tilting. The kneading motion, together with the pressure applied to the treatment region 330 by the head of transducer 310, presses against and squeezes the stored fat layer, which now contains dissolved fat molecules, triglycerides and toxins. For example, small circular massage motions can be interspersed with left-right-left tilting massage motions, or any combination of the above mentioned massage techniques, or other massage techniques known in the art. As a result, the internal pressure within fat cells 403 becomes greater than the external pressure (similar to a balloon). The pressure generated by the massage of the transducer 312 compresses fat layer 320 and the fat cells 403 therewith, and stimulates the triglycerides to be extracted from fat cells 403. It is noted that the kneading action or the pressure exertion, according to the disclosed technique, deviates from the general practice of applying ultrasound waves, which discourages any forceful contact between an ultrasound transducer and the skin.

A further measure to exert pressure on the treated region is by a manual or mechanical external massage, such as by massaging device 210. A practical and simple type of massage is the mere massaging by the bare hands of a treating person. However, other mechanical massaging means are effectively applicable. The massage presses downwards, as represented by arrows 414, against body region 330, and thereby squeezes some of the fat molecules, toxins and triglycerides out of the fat cells and stored fat layer 320. If a greater amount of fat 320 is present in body region 330, then a more intense massage is required. It is preferable to simultaneously apply the massaging action to the exact area on body region 330 where ultrasound waves 420 are applied, for achieving optimal effect. It is believed that the applied ultrasound softens the fat 320 tissue. Throughout the course of the process, a massage (e.g., using massaging device 210) also improves blood flow and operation of the lymphatic system. The massage can be applied effectively while the ultrasound is provided, or for a while thereafter. The entire duration of a single treatment session generally varies from about 15 to 45 minutes.

According to another aspect of the disclosed technique, electrical stimulation is applied to the treated region, causing the muscles in muscle layer 316 to alternately contract and relax. Electrodes 308 are attached to skin layer 313 with the aid of attaching means, such as adhesive patches, at the beginning and end of the muscle fibers that cross body region 330. Current is applied to body region 330 through electrodes 308 at frequencies ranging from 5-150 Hz, to stimulate intermittent contractions of the muscles. These contractions create a tense bedding of muscle against fat layer 320, providing an opposing force against the fat tissue being treated. The rapid contraction-relaxation motion of the muscles (represented by pressure arrows 409 in FIG. 4) repetitively presses against fat layer 320 from one direction, and squeezes out the fat molecules, toxins and triglycerides. Electrical stimulation can be applied while the ultrasound waves are being emitted, thereby enhancing the effectiveness of the fat extraction process, as the body region is further subjected to a strong mechanical pressure. In addition, application of pressure after exposure to ultrasound treatment is likewise effective for extracting fat. During the period of time immediately after the ultrasound treatment of the disclosed technique, and for a subsequent duration, fat cells 203 in treated region 330 have increased internal pressure, and some toxins, fat molecules and triglycerides have already been stimulated outside of the fat cells. It is believed that periodic application of pressure pulses with alternating relief intermissions is preferable over constant pressure application with respect to the tenability of living organic tissue, especially in circumstances of force accompanying an aggressive treatment. Accordingly, electrical stimulation as described hereinabove has been found to be effective for at least half an hour after an intensive ultrasound treatment in accordance with the disclosed technique.

The electrical stimulation of the muscles is preferably performed with interferential stimulation, such as: premodulated, biphasic, interferential isoplanar (4 poles), interferential vectorial (4 poles), and medium frequency stimulation techniques, as known in the art. The electrical stimulation is applied at intensities ranging from 5 to 90 mA. The interferential technique uses two alternating currents originating at different channels, each at slightly different carrier frequencies. These currents meet at treatment region 330 and create interference (constructive or destructive), producing a resultant beat frequency. The beat frequency is the difference between the actual frequencies provided by each pair. For example, a frequency of 100 Hz is yielded by 3,900 Hz in one electrode pair and 4,000 Hz in the other electrode pair. Accordingly, the resultant wave is a 3,900-4,000 Hz carrier wave modulated at an envelope amplitude frequency of 100 Hz. The dominant carrier frequency depends on the geometrical locations of the electrodes. Interferential stimulation is almost exclusively delivered with the quad-polar technique, in which four independent pads are placed in such a way as to achieve the desired effect. Typically, two pairs of electrodes are arranged around the treatment area, with each pair perpendicular to the other. Bipolar electrode placement may also be used, where the interference occurs within the generator rather than within the tissues, thereby requiring only one pair of electrodes to be used. The premodulated technique involves superimposing a signal with the effective frequency onto a continuously transmitted carrier wave, for instance, a 4000 Hz carrier wave modulated at an envelope amplitude frequency of 100 Hz. The modulation occurs before application thereof to a single pair of electrodes, rendering another pair of electrodes unnecessary. It is noted that multiple electrical stimulation techniques can be used, in various combinations, in various orders, and with various intermission durations (in between different electrical stimulation techniques), in accordance with the disclosed technique. For example, electrical stimulation during treatment may involve using the IF vectorial technique first for 10 minutes, then switching to the interferential technique for 5 minutes, then switching to the premodulated technique for an additional 5 minutes, then switching to the biphasic technique for another 10 minutes, then cycling back through this process again. According to another example, electrical stimulation during treatment may involve using the interferential technique first for 8 minutes, then switching to the IF vectorial technique for 2-3 minutes, then switching to the premodulated technique for 6 minutes, then to the biphasic technique for 7 minutes, then switching to the MF stimulation for 5-10 minutes then cycling back through this process again. It was found that the interferential, IF isoplanar, IF vectorial, and premodulated techniques are preferable, with the IF vectorial technique being the most effective in terms of stimulating muscle contractions to aid in squeezing out triglycerides from the fat cells. While each electrical stimulation technique is applied, the carrier wave frequency is preferably changed (hopped) at least once, thus avoiding adaptation of the body to the stimulation (thereby ceasing to react with intermittent contractions), and avoiding the need to increase the stimulation intensity. For example, while each electrical stimulation technique is applied, the carrier wave may be hopped from a 4,000 Hz carrier wave to a 2,400-2,500 Hz carrier wave. Similarly, the envelope or beat frequency (where relevant) is changed gradually or hopped between selected frequencies.

During the initial treatment session, it is preferable to use low current intensities in the range of approximately 3-5 mA, as a higher current intensity can be agitating and may alarm an inexperienced patient. In more advanced treatments, it is possible to apply the more effective higher current intensities in the range of approximately 5-90 mA. The effective frequencies are between approximately 5-150 Hz. It is noted that when using the different electrical stimulation techniques mentioned above, for example interferential, premodulated, and the like, the muscles do not react (with intermittent contractions) to frequencies above approximately 250 Hz. At higher frequencies the vibrations are so frequent that the muscles can remain constantly tense. At lower frequencies, the vibrations are slower but much stronger. Since the muscle adapts to a specific frequency, it is therefore advisable to alter the frequency of the electrical stimulation throughout the duration of the treatment, and even during a specific stimulation technique. Apart from using a specific frequency and/or altering the frequency manually or arbitrarily, other patterns for altering frequency include the following: (1) applying a specific frequency for a fixed amount of time before switching to another frequency; (2) gradual change, such as going from 5 to 150 Hz and back (such as in a sinusoidal cycle); (3) similar to pattern (2), but remaining for a longer duration (such as 1 second) at the extreme levels; (4) only the extreme frequencies are used intermittently. Other patterns for altering the frequency may also be used.

According to another aspect of the disclosed technique, a gel 309 is rubbed onto skin layer 313 at treatment region 330 prior to ultrasound application. Gel 309 is preferably water-based, to conform to the ultrasound conductive medium. Preferable gels can include ingredients such as: hydroxyl acids, plant extracts, wheat proteins, macadamia oil, chamomile, zinc, salicylic acid, and caffeine. Gel 309 has several purposes. Firstly, gel 309 effectively conducts ultrasound waves 402 between the ultrasound transducer 312 and tissues in body region 330. Gel 309 is also designed to provide smooth penetration of the ultrasound waves 402 to the underlying tissues. In addition, gel 309 lubricates the skin and prevents friction and scrapes to the skin, especially in circumstances where the head of ultrasound transducer 312 is strongly rubbed in a kneading or similar motion to provide a massage to body region 330. Also, drugs and active ingredients, if added to gel 309, are absorbed into the epidermis layer (skin layer 313) more effectively because of ultrasound waves 402, the heated fluids and tissue material, and the appearance of ruptures or cracks in body region 330. This absorption is further enhanced by the head of ultrasound transducer 312 forcefully rubbing gel 309 against the skin. The drugs or active ingredients that are absorbed may catalyze blood flow and circulation, and transmit into the skin surface, and perhaps beneath, minerals and nutrients that the skin may lack. The application of the nutrients can also substantially improve the skin appearance. Throughout the course of the process, the massage action that rubs gel 309 into the skin improves blood flow and operation of the lymphatic system.

The application of one or any combination of any of the four pressure increasing measures detailed above (i.e., micro-massage, ultrasound transducer kneading, electrical stimulation, and manual/mechanical massage), can exert sufficient and suitable pressure on body region 330 from one direction (e.g. from above if the patient is lying down supine and the body region is the patient's stomach) and from an opposite direction (e.g., from below in the example aforementioned), that contributes to an effective treatment. It was found that by applying a greater number of the aforementioned pressure increasing measures, a more substantial reduction of stored fat occurs. The micro-massage is an outcome of the ultrasound application. The optional ultrasound transducer kneading, the electrical stimulation, and the manual/mechanical massage can be applied simultaneously with the ultrasound application, as well as for a period thereafter. It is conjectured that the exertion of pressure by ultrasound transducer kneading and/or manual/mechanical massage from one direction, together with electrical stimulation from an opposite direction, most effectively promotes squeezing out the fat molecules, toxins and triglycerides from fat layer 320.

In addition, an improvement in the circulatory system and metabolism processes of the body is anticipated to result from the treatment of the disclosed technique. Due to the breakdown and release of the triglycerides and toxins, the arteries and capillaries in the blood between fat cells that were previously narrowed by the enlarged fat cells (i.e., vasoconstriction), now become widened (i.e., vasodilation). Blood circulation is then accelerated, and the tissues receive more oxygen and nutrients. This in turn improves the purification process and tissue regeneration in the body. As a result, the blood system and lymphatic system return to their normal states. This development helps remove the extraneous stored fat from the body.

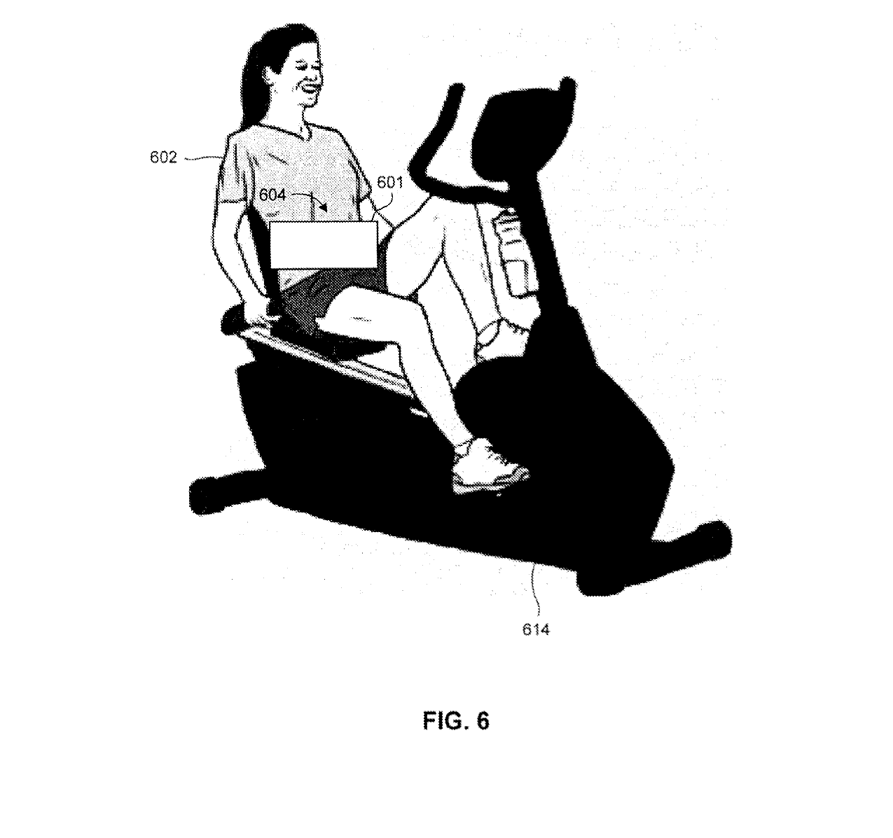

According to a further aspect of the disclosed technique, system 200 is applied to a person who is performing a physical exercise. Reference is now made to FIG. 6, which is a schematic illustration of an ultrasound apparatus, referenced 601, used during a treatment session while the treated person is exercising, constructed and operative in accordance with another embodiment of the disclosed technique. Ultrasound apparatus 601 is applied to the abdominal region 604 of a person 602 who is riding an exercise bicycle 614. Research performed (such as in a study elaborated herein below) has supported the initial premise that exercise shortly after the ultrasound treatment, optionally in conjunction with electric stimulation and pressure exertion, significantly enhances fat reduction, by accelerating the relative proportion of stored fat catabolism, particularly in the treated body region.

As discussed hereinabove, the applied ultrasound generates heat and pressure within fat cells in the treated body region, encouraging the release of triglycerides, dissolved fats and toxins from the fat cells. Consequently, the released triglycerides and dissolved fats are broken down in metabolic pathways associated with catabolism in order to extract energy for the body, or alternatively, are removed as waste products or are reabsorbed into cells and tissues. Accordingly, catabolism of the fat cells is carried out at a higher proportion than otherwise would have been. It has been experimentally shown that exercise shortly after the ultrasound application significantly increases the proportion of triglycerides relative to carbohydrates that are converted into energy, as well as speeds up circulatory processes in the body, causing the circulatory system to more effectively cleanse the body by collecting and removing waste, which may include the released triglycerides, fat molecules and toxins. It is also believed that a body that is well hydrated during or immediately after the applied ultrasound further accelerates the relative proportion of stored fat catabolism in the body.

Ultrasound apparatus 601 may treat different regions of the body, including thighs, buttocks, stomach, upper abdomen, back, neck, arms, legs, and the like. Ultrasound apparatus 601 may be portable and have different forms that may include a vest, trousers, and arm or thigh bands, to enable ultrasound apparatus 601 to fit snugly or tightly onto the treated body region (e.g., abdominal 604) while facilitating performance of the exercise in conjunction with the ultrasound application. It is noted that a portable ultrasound apparatus may use a gel encased between treatment region 604 and the ultrasound apparatus 601 transmitting the ultrasound waves (similarly to gel 309 described hereinabove). It is appreciated that exercise bicycle 614 may generally be any type of exercising equipment, such as a bicycle, a treadmill, a stepper machine, an elliptical trainer, a jump rope, free weights, and the like. Accordingly, the exercise may take on many forms, such as riding an exercise bike, lifting weights, running on a treadmill, performing calisthenics, jumping rope, performing lifts, or other physical activities. The exercise activities may be performed in standing, sitting, or lying down (prone or supine) positions, depending on what is most comfortable based on the targeted body region and the individual undergoing the treatment.

The exercise may be performed over a range of exercise intensities and durations. A preferable exercise intensity level is approximately 60% of maximum heart rate (i.e., 220 minus age in years). It is considered that this exercise intensity level results in an optimal amount of fat relative to carbohydrates being used by the body (i.e., undergoing catabolism) during exercise, and is also an exercise intensity level that is relatively easily attainable for most patients. If a patient is sufficiently fit and wants to increase his/her targeted fat reduction goals, it is recommended that the patient perform the exercise for a longer duration, rather than at a higher intensity, for more effective fat reduction (via the accelerated relative proportion of stored fat catabolism).

It will be appreciated that the disclosed technique may also result in the reduction or elimination of the common cellulite condition. Additionally, the disclosed technique may also be effective in reducing body contour and smoothing skin in general.

The system of the disclosed technique may be adapted for personal use by an individual, such as at his/her home or at any convenient location, without necessitating a visit to a clinic or office in order to be treated by another person.

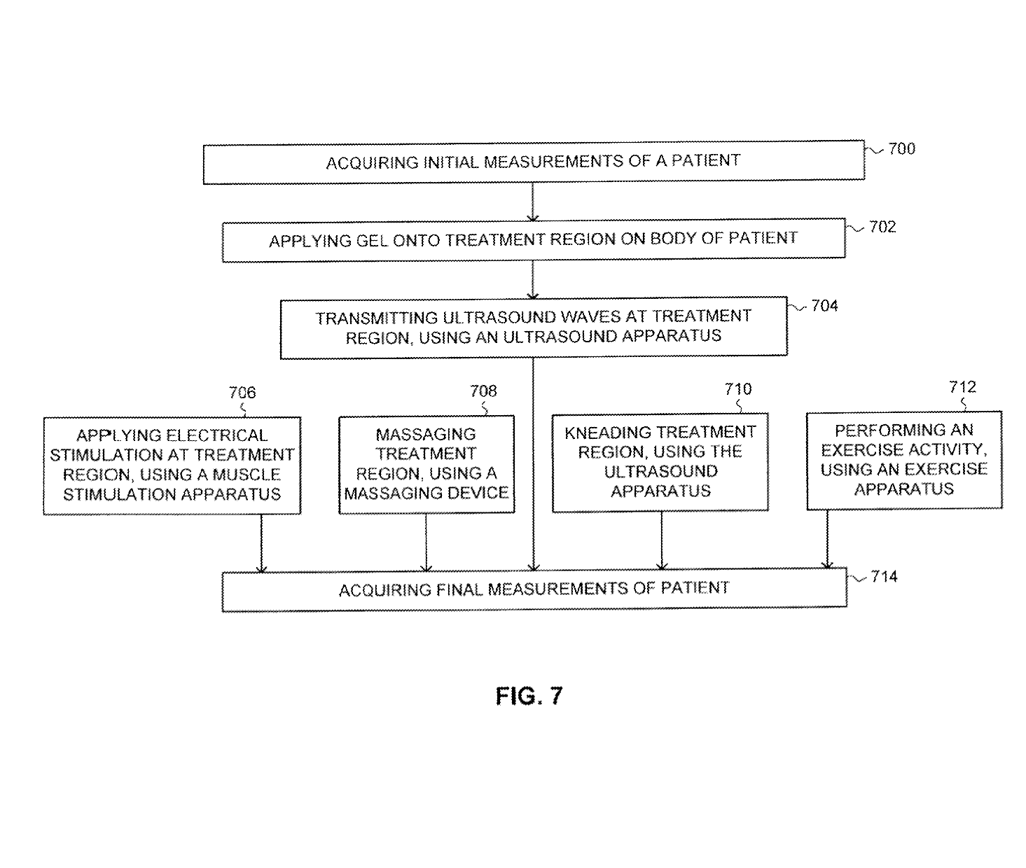

Reference is now made to FIG. 7, which is a flow diagram of a method for accelerating the relative proportion of stored fat catabolism, operative in accordance with an embodiment of the disclosed technique. In an optional procedure 700, initial measurements of a patient are acquired. Referring to FIG. 2, patient measurement apparatus 204 measures physical parameters of the patient such as weight, interior or exterior contour of a body part, body fat ratio, and the like. The measured physical parameters may be recorded.

In an optional procedure 702, gel is applied onto a treatment region on the body of the patient. With reference to FIGS. 3 and 4, gel 309 is applied to skin layer 313 at body region 330, to facilitate the penetration of ultrasound waves 402 to fat layer 320.

In procedure 704, ultrasound waves are transmitted at the treatment region using an ultrasound apparatus. With reference to FIGS. 3 and 4, ultrasound transducer 312 transmits ultrasound waves 402 at body region 330.

In an optional procedure 706, electrical stimulation is applied to the treatment region using a muscle stimulation apparatus. With reference to FIGS. 3 and 4, electrodes 308 are attached to the body of the treated person at body region 330, and a current is applied through electrodes 308 to stimulate contractions of muscles 316. The contracting muscles press against the fat cells in fat layer 320, further facilitating the release of triglycerides and dissolved fats from the fat cells.

In an optional procedure 708, the treatment region is massaged using a massaging device. Referring to FIG. 3, massaging device 210 massages and applies pressure to body region 330, further facilitating the release of triglycerides, fat molecules, toxins from the fat cells in fat layer 320.

In an optional procedure 710, the treatment region is kneaded using the ultrasound apparatus. Referring to FIGS. 3 and 4, ultrasound transducer 312 applies a kneading motion onto body region 330 while transmitting ultrasound waves 402, applying pressure on body region 330 and further facilitating the release of triglycerides, fat molecules, toxins from the fat cells in fat layer 320.

In an optional procedure 712, an exercise activity is performed using an exercise apparatus. With reference to FIGS. 2, 3, 4 and 6, the patient uses exercise apparatus 214, such as exercise bike 614, to perform exercise while ultrasound waves 420 are being applied to body region 330. Alternatively, the exercise may be performed immediately after the application of ultrasound waves 420. The exercise accelerates the catabolism of the released triglycerides in order to extract energy for the body, and consequently the effective reduction of stored fat.

The treated body region is preferably subjected to the ultrasound application (procedure 704) at the same time as the applied electrical stimulation (procedure 706), in order to facilitate and enhance the release of triglycerides, fat molecules and toxins from the fat cells in the body region. Alternatively, procedure 706 can be performed after procedure 704, or further alternatively, both during and after procedure 704. Regardless when procedure 706 is performed, if the frequency or intensity of the electrical stimulation is varied rapidly, it is then suggested that the frequency of the applied ultrasound be varied slowly. Conversely, if the frequency or intensity of the electrical stimulation is varied slowly, it is then suggested that the frequency of the applied ultrasound be varied rapidly. In other words, it is suggested that the rate of varying the parameters related to the electrical stimulation (procedure 706) (frequency and intensity) should be inversely proportional to the rate of varying the parameters related to the applied ultrasound (procedure 704) (frequency). Alternatively, the rate of varying the parameters of both the applied electrical stimulation and the applied ultrasound may be proportional and even identical.

Preferably, the massage (procedure 710) is applied to the treated body region concurrently with the electrical stimulation (procedure 706) and the ultrasound application (procedure 704), but may alternatively be applied after the ultrasound application.

The exercise (procedure 712) is preferably undertaken immediately after, but also optionally in conjunction with the ultrasound application (procedure 704) and also optionally with the electrical stimulation (procedure 706), with the massage (procedure 708), and with the kneading (procedure 710)

If procedures 706 or 708 are carried out after procedure 704, they are preferably carried out simultaneously, irrespective of the fact that any of procedures 706 and/or procedure 708 was already applied or not applied also during procedure 702. A post-ultrasound massage (procedure 708) or electrical stimulation (procedure 706) or the preferable combined massage and electrical stimulation in a post-ultrasound stage, requires 20-30 minutes. Further preferably, massage (procedure 708) or electrical stimulation (procedure 706), or exercise (712) is carried out both during and after the ultrasound application (procedure 704). Optionally, both the massage (procedure 708), the electrical stimulation (procedure 706), and exercise (procedure 712) are performed together both during and after the ultrasound application (procedure 704). With regard to the duration of the treatment session, the 20-30 minutes of post-ultrasound stage (procedures 706 and 708) and the 30 minutes of post-ultrasound stage (procedure 712) follows the initial 45 minutes stage devoted to a combined ultrasound, massage, stimulation and exercise stage (procedures 704, 706, 708, 710 and 712).

In an optional procedure 714, the final measurements of the patient are taken. With reference to FIG. 2, patient measurement apparatus 204 measures physical parameters of the patient. The final measurements are compared with the initial measurements, to ascertain the degree of change in the patient as a result of the treatment.

Several treatment sessions may be required to achieve satisfactory results. In summary, the treatment is a combination of several elements: application of a gel, application of ultrasound waves, ultrasound transducer kneading, electrical stimulation of the muscles, manual or mechanical massage, and exercise.

A study performed at the Hadassa Hospital Sport Medicine Center in Jerusalem, Israel, designed by Dr. Naama Constantini, Director of Sport Medicine Center, concluded that “a single ultrasound treatment of the disclosed technique increased the percentage of fat used as an energy source during moderate-intensity exercise by 52% compared with pre-treatment values, an increase which was significantly higher than that of the sham treatment. This finding may support the hypothesized fate of subcutaneous lipids released by the disclosed technique, which is that they oxidized as an energy source”. The study was approved by the institutional review board for medical ethics of the Hadassah Medical Organization. All participants signed the informed consent forms. A further elaboration of the study is included herein as Appendix A.

The study performed single blind tests comparing the effects of the disclosed ultrasound treatment with a placebo treatment applied in conjunction with moderate-intensity exercise, immediately following treatments. In moderate-intensity exercise, there is an increase in oxidation of both carbohydrates and fat. High-intensity exercise utilizes mainly carbohydrate as the energy source. It is known that at the moderate exercise intensity of about 45-65% of the maximal exercise capacity, fat oxidation rate is maximal. An increase in fat oxidation following an ultrasound treatment is most apparent under maximal utilization rates, i.e., during exercise at moderate intensity.

The source of energy used during resting or exercise conditions is determined by measuring oxygen (O₂) and carbon dioxide (CO₂) gas exchange by a metabolic system. The ratio between CO₂ and O₂ in the expired air, named the Respiratory Quotient (RQ, or R), is analogous to the ratio between carbohydrates and fat as energy sources.

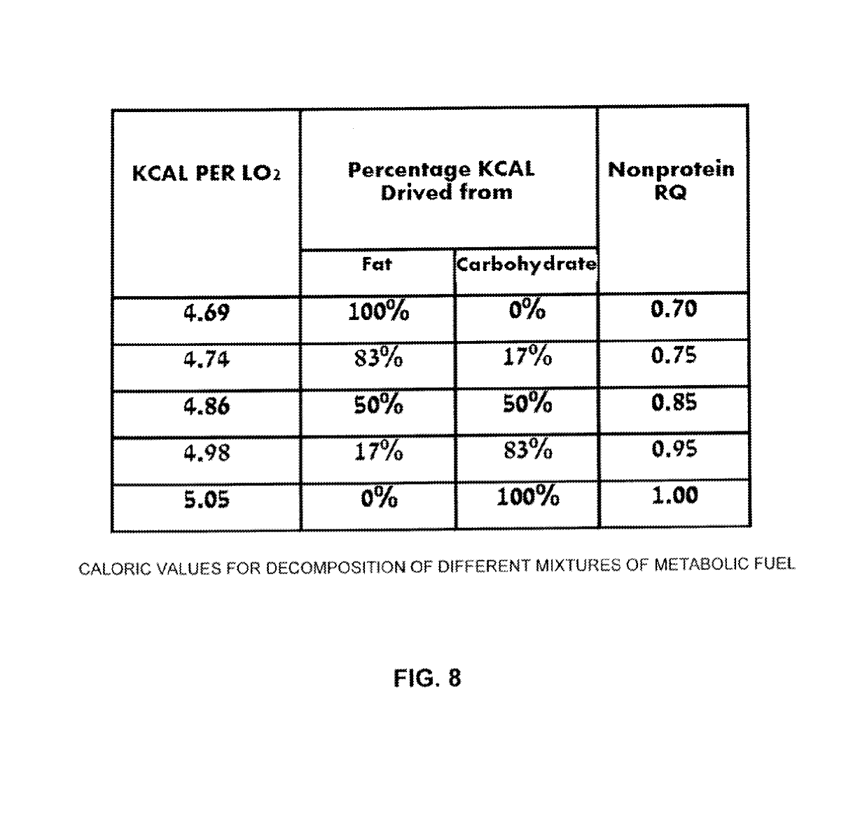

Reference is now made to FIG. 8, which is a table illustrating the relation between (non-protein) R values, KCAL (Kilo-calorie) percentage derived from fats and carbohydrates, respectively, and KCAL per L0₂. In general, an R value of 1.0 is obtained when carbohydrates are the sole source of energy, while an R value of 0.7 is obtained when fat is the sole source of energy. During rest or (moderate exercise, intermediate values of R are obtained, ranging between these two extremes (i.e., between 0.7 and 1.0), depending on the ratios of oxidation of carbohydrates and fat. Lower values, closer to 0.7, signify higher fat oxidation, while higher values closer to 1.0 suggest a larger contribution of carbohydrates as the muscle's energy source. For example, an R value of 0.85, which is halfway between 0.7 and 1.0, suggests that half of the energy derives from carbohydrate oxidation and half from fat for the current activity.

Measuring gas exchange and calculating the R value before and after ultrasound treatment of the disclosed technique, can demonstrate whether there has been a change in fat oxidation following the treatment.

The study was performed on randomly selected volunteer females, with ages ranging from 18 to 40, and with a body mass index (BMI) above 25 kg/m² (which indicates an overweight status).

The first phase of the experiment established a baseline R value for moderate-intensity exercise level before ultrasound treatment. During the first phase, the participants walk on a treadmill at a gradually increasing speed and inclination, until reaching the intensity at which the heart rate was at 60% of predicted maximal heart rate (i.e., 220 minus age in years). This baseline exercise level was maintained for 15 minutes, and was recorded and adhered to in the later phases of the experiment.

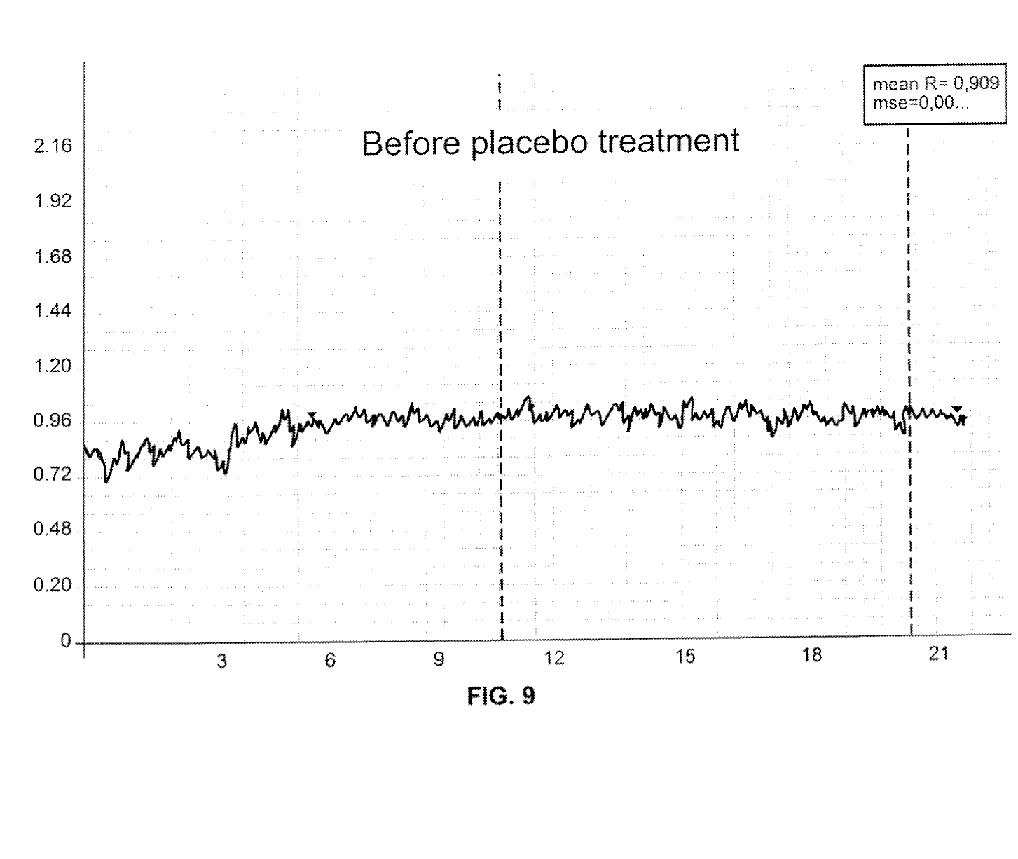

Reference is now made to FIG. 9, which is a graph illustrating R values as a function of exercise time prior to ultrasound treatment, for a first participant in an experimental study for the disclosed technique. The baseline R value for this participant was approximately 0.91. The baseline R value is calculated as the mean respiratory gas exchange during the last 10 minutes of exercise, which is measured using a face mask connected to a validated portable metabolic system (COSMED K4b2, Rome, Italy).

In the next phase of the experiment, participants underwent either a genuine treatment (in accordance with the disclosed technique) or a placebo treatment, to the abdominal area, without the participants knowing which type of treatment they were undergoing. After the treatments, the baseline exercise session was repeated, and the R values were determined.

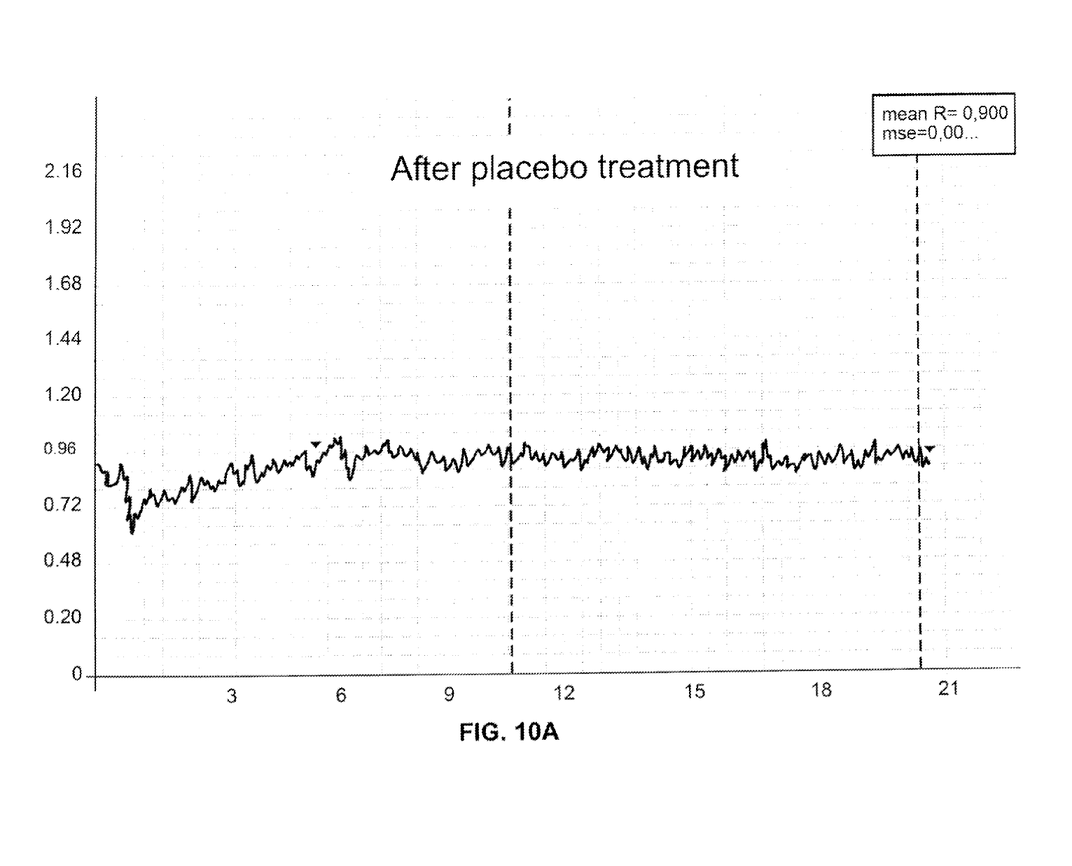

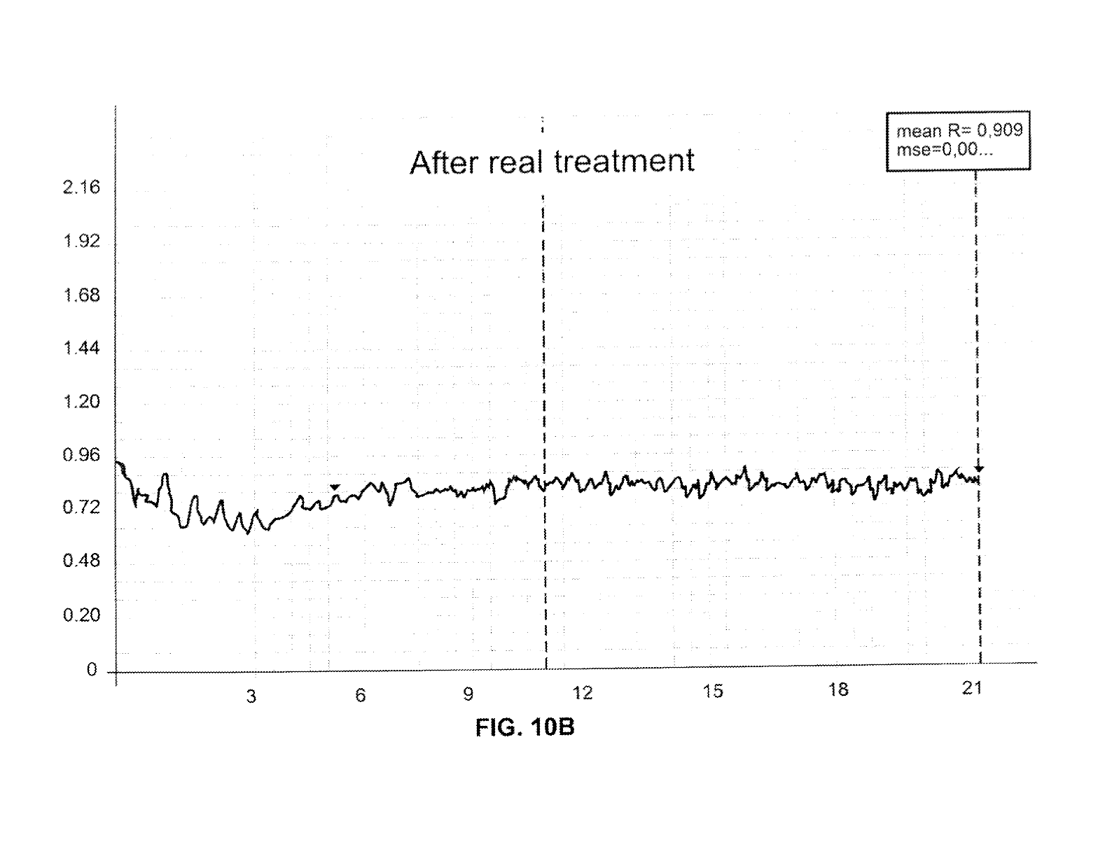

A few days later, the participants returned and all testing procedures were replicated, except that participants were switched testing groups (i.e., those participants who earlier underwent the genuine treatment now underwent the placebo treatment, and vice-versa). Reference is now made to FIGS. 10A and 10B. FIG. 10A is a graph illustrating the R values as a function of exercise time for the first participant in an experimental study for the disclosed technique, after undergoing a placebo treatment. The graph of FIG. 10A shows that the mean R value for this participant decreased to 0.90, indicating a small increase in fat consumption. FIG. 10B is a graph illustrating the R values as a function of exercise time for a second participant in an experimental study for the disclosed technique, after undergoing an ultrasound treatment in accordance with the disclosed technique. The graph of FIG. 6B shows that the mean R value decreased to 0.79, indicating a significant increase in fat consumption.

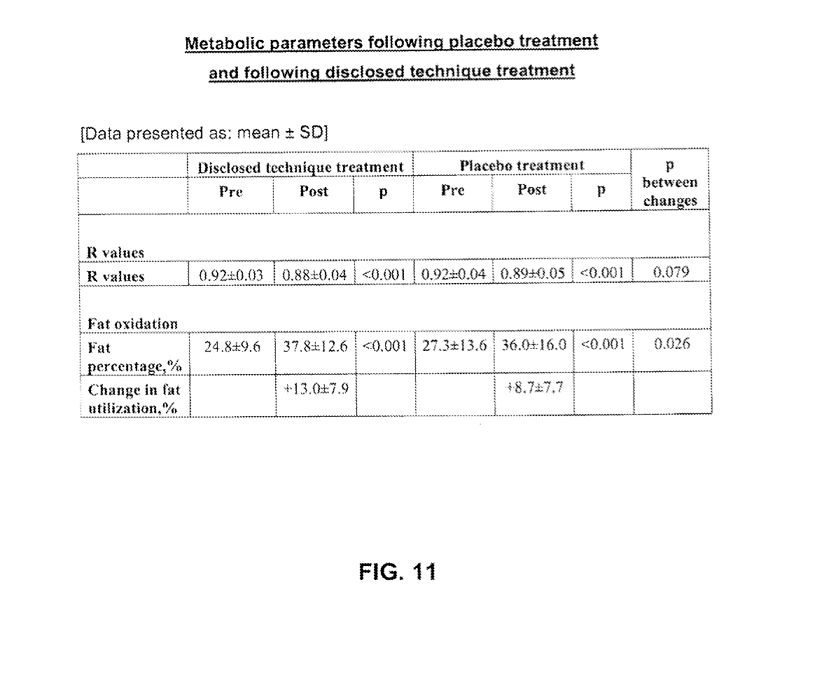

Reference is now made to FIG. 11, which is a table summarizing the quantitative metabolic results for all the participants in an experimental study for the disclosed technique. The Wilcoxon signed-ranks test was used in order to compare between pre-treatment and post-treatment measurements in each session. Spearman's correlation was used to measure the relationships between the clinical/anthropometric parameters and the changes in R values or the percentage of fat utilization. FIG. 11 presents the R values and the corresponding proportion of fat as an energy source during the exercise sessions. Heart rates during all exercise sessions were similar, and slightly above the desired range of 60% of the maximal heart rate. Post-placebo treatment R values were slightly lower, and the proportion of fat as an energy source was higher by 32% at post-test compared with pre-test values. Post-treatment R values were significantly lower, and the proportion of fat as an energy source was higher by 52% at post-test compared with pre-test values. The differences between the observed increases in fat utilization following the placebo and ultrasound treatment of the disclosed technique was statistically significant (p=0.02). No correlations were found between age, weight, height or BMI and the differences in R or the percent of fat used in exercise. With 21 participants, the study had a power of 94.8% to detect the observed difference.

This study demonstrated that a single ultrasound treatment of the disclosed technique increased the percentage of fat used as an energy source during moderate-intensity exercise by 52% compared with pre-treatment values, an increase which was significantly higher than that of the placebo treatment. It is noted that this finding may support the hypothesis that the subcutaneous fat (fat layer 320 of FIGS. 3 and 4) released by the ultrasound treatment of the disclosed technique is oxidized as an energy source.

It will be appreciated by persons skilled in the art that the technique is not limited to what has been particularly shown and described hereinabove.

### APPENDIX A

A Single Treatment with the Du857 Ultrasound and Interferential Unit (“Bella Contour” Unit) Increases Fat Oxidation During Exercise

### Introduction

The DU857 (“Bella Contour”, BC) is a non-invasive device that uses ultrasonic resonant technology in order to break subcutaneous fat cells and reduce the circumference of the treated body part, to smooth the skin, and to reduce cellulite. The device operates by identifying the location of the subcutaneous fat via transmission and reception of ultrasonic waves, in a manner similar to that of other ultrasound diagnostics identification systems. The system identifies the adipose tissue layer's thickness, depth and fatty-tissue density. It then performs a mathematical calculation of the frequency and intensity of a hypersonic wave that, when transmitted, would cause the fat cells to vibrate and enter into self-resonance. The combined vibration and self-resonance of the fat cells generates warmth and breaks down the cell membranes, thus releasing lipids to the reticular tissue. This process has been verified by histologic studies, yet the fate of this released fat is unknown. The clinical experience with BC treatments is that there are indeed reductions in the circumference of treated areas, suggesting a mobilization of the adipose tissue. It has been speculated that the lipids released by BC treatment are oxidized as an energy source—but this has not been previously examined. The two main energy sources of the active muscle are carbohydrates and fat [McArdle W D, Katch F I, Katch V L. Exercise Physiology: Energy, Nutrition, and Human Performance. 6^{th} ed. 2006. Lippincott Williams & Wilkins. Md, USA]. During exercise, both of these sources can contribute to the increased energy demand. Low-intensity exercise uses fat as the main source of energy, but in small absolute amounts due to the small needs. In moderate-intensity exercise there is an increase in oxidation of both carbohydrates and fat. High-intensity exercise utilizes mainly carbohydrates as the energy source. It is known that at the moderate exercise intensity of about 45-65% of the maximal aerobic capacity, the fat oxidation rate is maximal [Achten J, Jeukendrup A E. Optimizing fat oxidation through exercise and diet. Nutrition. 2004; 20:716-27]. An increase in fat oxidation following BC treatment would be most apparent under maximal utilization rates, i.e. during exercise at moderate intensity. In addition, as this exercise intensity is recommended to maintain general health [U.S. Department of Health and Human Services. 2008 Physical Activity Guidelines for Americans] and is fairly easy to perform, demonstrating increased fat oxidation at this specific exercise level may also carry a practical message.

The source of energy used during resting or exercise conditions is determined by measuring oxygen (O₂) and carbon dioxide (CO₂) gas exchange by a metabolic system [McArdle W D, Katch F I, Katch V L. Exercise Physiology: Energy, Nutrition, and Human Performance. 6^{th} ed. 2006. Lippincott Williams & Wilkins. Md, USA]. The ratio between CO₂ and O₂ in the expired air, named the Respiratory Quotient (RQ, or R), is analogous to the ratio between carbohydrates and fat as energy sources. In general, an R value of 1.0 is obtained when carbohydrates are the sole source of energy; an R value of 0.7 is obtained when fat is the sole source of energy. During rest or sub-maximal exercise, intermediate values of R are seen ranging between these two extremes, depending on the oxidation ratios of carbohydrates and fat. Lower values, closer to 0.7, signify higher fat oxidation, while higher values closer to 1.0 suggest a larger contribution of carbohydrates as the muscle's energy source. For example, an R value of 0.85, which is halfway between 0.7 and 1.0, suggests that half of the energy for the current activity derives from carbohydrate oxidation and half from fat. Measuring gas exchange and calculating the R value before and after BC treatment, can demonstrate whether there has been a change in fat oxidation following treatment.

The aim of this study was to assess whether a single BC treatment causes a change in lipid oxidation during moderate-intensity exercise.

### Methods

### Study Population

The inclusion criteria for study participation was female sex, age 18-40, with a body mass index (BMI) above 25 kg/m², which indicates an overweight status and corresponds with the main clinical target population. The presence of any underlying disease/illness, use of chronic medications, pregnancy, smoking, or a past BC treatment were the exclusion criteria. Participants were recruited by advertisements sent via intra-organizational emails to all 6,000 employees of the Hadassah Medical Center, or by inviting women who called and requested details regarding BC treatments. The study was approved by the institutional review board for medical ethics of the Hadassah Medical Organization. All participants signed the informed consent forms.

### Study Protocol

Study participants came to the BC clinic at “Hadassah Optimal” Medicine Center on two occasions, during the morning hours.

In the first visit, inclusion and exclusion criteria were verified, the study protocol was explained, questions were answered, and informed consent forms were signed. Height and weight were measured using standard equipment (height was rounded to the nearest 1 cm, weight was rounded to the nearest 0.1 kg), and BMI was calculated. Then the participant underwent the first exercise session, under the supervision of an exercise physiologist from the “Hadassah Optimal” Sport Medicine Center. The participant walked on a treadmill at a gradually increasing speed and inclination until reaching the intensity at which the heart rate was at 60% of the predicted maximal heart rate (i.e., 220 minus age in years). The speed and grade were recorded, and these parameters were used for performing the following moderate-intensity exercises, before and after the BC and sham treatment sessions. As a baseline exercise test, the study participant walked for 15 minutes while connected to the metabolic system, at the intensity previously identified which corresponds to 60% of maximal heart rate. Respiratory gas exchange measurements were performed using a face mask connected to a validated portable metabolic system (COSMED K4b2, Rome, Italy [McLaughlin J E, King G A, Howley E T, Bassett D R Jr, Ainsworth B E. Validation of the COSMED K4 b2 portable metabolic system. Int J Sports Med. 2001; 22:280-4]), and were used to determine the baseline R value. The R value used for calculation purposes was the mean of the last 10 minutes of exercise. The heart rate during exercise was also calculated as a mean of the last 10 minutes of exercise.

After exercise termination, the participant was escorted to the BC treatment room where a BC technician and the study coordinator performed the treatments. Participants underwent either a real BC treatment to the abdominal area or a sham one. The order of treatments was determined randomly by the study coordinator, which selected one of two paper notes from a sealed bag that designated the first treatment to be real or sham. The sham treatment consisted of the same measurements and treatment movements as in the real one, but without ultrasound emission. After treatment, the exercise session was repeated, and the R value after this single BC treatment (real or sham) was calculated. No eating was allowed during the whole visit, only the drinking of small amounts of water if desired.

In the second visit, which was held after a range of 3-7 days, all testing procedures were replicated. The treatment applied was either real or sham, depending on the type of treatment given at the first visit, so each participant single-blindly underwent one real and one sham procedure.

### Statistical Analysis

The Wilcoxon signed-ranks test was used in order to compare between pre and post-test measurements in each session. Spearman's correlation was used to measure the relationships between the clinical/anthropometric parameters and the changes in R values or the percentage of fat utilization.

### Results

The demographic and anthropometric characteristics of the 21 study participants and the exercise parameters are presented in Table 1. All participants were overweight (BMI>25 kg/m²), as per study inclusion criteria.

TABLE 1
Study participant characteristics

| | Mean ± SD | Range |
|---|---|---|
| Age (yrs): | 31.4 ± 5.2 | 21-40 |
| Height (cm): | 161 ± 5 | 152-171 |
| Weight (kg) | 78 ± 7.5 | 63.5-88.4 |
| BMI (kg/m²) | 30.1 ± 2.4 | 26.5-34.8 |
| Exercise load | | |
| Treadmill speed (km/h) | 4.3 ± 0.3 | 4-5 |
| Treadmill grade (%) | 2.3 ± 1.0 | 0-4 |

Table 2 presents heart rates, the R values, and the corresponding proportion of fat as an energy source during the exercise sessions. Heart rates during all exercise sessions were similar, and slightly above the desired range of 60% of the maximal heart rate. Post-sham treatment R values were significantly lower, and the post-test proportion of fat as an energy source was 32% higher than pre-test values. Post-treatment R values were significantly lower, and the post-test proportion of fat as an energy source was 52% higher than pre-test values. The differences between the observed increases in fat utilization following the sham and real BC treatments was statistically significant (p=0.02). No correlations were found between age, weight, height or BMI and the differences in R or the percent of fat used in exercise. With 21 participants, the study had a power of 94.8% to detect the observed difference.

TABLE 2
Metabolic and exercise parameters following sham and
real BC treatments (Data presented as mean ± SD)

| BC Treatment | | | Sham Treatment | | | p between |
|---|---|---|---|---|---|---|
| Pre | Post | p | Pre | Post | p | changes |

Exercise Heart Rate

| | | | | | | | |
|---|---|---|---|---|---|---|---|
| HR (bpm) | 125 ± 12 | 127 ± 12 | 0.423 | 125 ± 10 | 126 ± 10 | 0.255 | — |
| HR (% of predicted | 66 ± 5 | 67 ± 5 | | 66 ± 5 | 67 ± 4 | | |
| maximal) | | | | | | | |

R values

| | | | | | | | |
|---|---|---|---|---|---|---|---|
| R values | 0.92 ± 0.03 | 0.88 ± 0.04 | <0.001 | 0.92 ± 0.04 | 0.89 ± 0.05 | <0.001 | 0.0079 |

Fat oxidation

| | | | | | | | |
|---|---|---|---|---|---|---|---|
| Fat percentage (%) | 24.8 ± 9.6 | 37.8 ± 12.6 | <0.001 | 27.3 ± 13.6 | 36.0 ± 16.0 | <0.001 | 0.0026 |
| Change in fat | — | +13.0 ± 7.9 | | — | +8.7 ± 7.7 | | |
| utilization (%) | | | | | | | |

### Discussion

This study demonstrated that a single BC treatment increased the percentage of fat used as an energy source in moderate-intensity exercise by 52% compared with pre-treatment values. This increase was significantly larger than that seen following the sham treatment. The effect was similar regardless of age, height, weight or BMI.

The target exercise intensity was verified by monitoring the heart rate, which was only slightly higher than the intended 60%, but resided within the area of the so called “fat burning zone” of heart rate [Carey D G. Quantifying differences in the “fat burning” zone and the aerobic zone: implications for training. J Strength Cond Res 2009; 23:2090-5].

The result that the sham treatment also caused a statistically significant increase in the rate of fat utilization during exercise, can be explained by previous data demonstrating that a rest period between two exercise sessions causes an increase in fat oxidation in the second session [Carey D G. Quantifying differences in the “fat burning” zone and the aerobic zone: implications for training. J Strength Cond Res 2009; 23:2090-5; Goto K, Ishii N, Mizuno A, Takamatsu K. Enhancement of fat metabolism by repeated bouts of moderate endurance exercise. J Appl Physiol 2007; 102:2158-64]. Therefore, the resting period between the exercise sessions is a probable explanation for this finding. This effect is also expected to occur after BC treatment, but the magnitude of change was significantly higher compared to that of a sham treatment.

### CONCLUSION

In conclusion, this study has demonstrated that a single BC treatment increased the percentage of fat used as an energy source during moderate-intensity exercise by 52% compared with pre-treatment values, an increase which was significantly higher than that of the sham treatment. This finding may support the hypothesized fate of subcutaneous lipids released by BC treatment, which is oxidation as an energy source.

1-43. (canceled)

44. A system for reducing stored body fats in a treatment region on the body of a treated person, the system comprising:
an ultrasound apparatus, operative for transmitting ultrasound waves at said treatment region, wherein said ultrasound apparatus is adapted to be worn on said treatment region by said treated person undergoing a physical exercise; and
an electrical stimulation apparatus, operative for applying electrical stimulation to said treatment region substantially concurrently to the transmission of said ultrasound waves, to stimulate intermittent contractions of the muscles at said treatment region,
wherein said treated person is undergoing a physical exercise substantially concurrently to the transmission of said ultrasound waves and to the application of said electrical stimulation,
whereby the combination of said transmission of said ultrasound waves, said application of said electrical stimulation, and said physical exercise, facilitates the release of triglycerides, fat molecules and toxins from stored fat within said treatment region, and accelerates the relative proportion of fat catabolism in said treatment region.

45. The system of claim 44, wherein said transmitted ultrasound waves comprises at least one selected from the list consisting of:
ultrasound waves at a frequency between 1-3 MHz;
ultrasound waves at a frequency which is altered according to a frequency variation pattern over the course of a treatment session; and
ultrasound waves with an intensity between 1.5-2.1 W/cm².

46. The system of claim 44, further comprising an exercise apparatus, used by said treated person to perform said physical exercise.

47. The system of claim 44, wherein said physical exercise is performed at an intensity level of approximately 60% of maximum heart rate.

48. The system of claim 44, wherein said electrical stimulation comprises at least one selected from the list consisting of:
interferential stimulation;
premodulated interferential stimulation;
biphasic interferential stimulation;
interferential isoplanar (4 poles) interferential stimulation;
interferential vectorial (4 poles) interferential stimulation;
medium frequency interferential stimulation;
current intensity between 5-90 mA;
electrical stimulation at a frequency between 5-150 Hz; and
electrical stimulation at a frequency which is altered according to a frequency variation pattern.

49. The system of claim 44, wherein pressure is exerted onto said treatment region substantially concurrently to the transmission of said ultrasound waves at said treatment region.

50. The system of claim 49, wherein said pressure is exerted by manually kneading a portion of said ultrasound apparatus against said treatment region.

51. The system of claim 44, further comprising a massaging device, operative for massaging said treatment region substantially concurrently to the transmission of said ultrasound waves at said treatment region.

52. The system of claim 44, further comprising a measurement apparatus, operative for measuring physical parameters of said treated person, substantially concurrently to the treatment.

53. The system of claim 44, further comprising a camera, operative for recording the treatment.

54. A method for reducing stored body fats in a treatment region on the body of a treated person, the method comprising the procedures of:
transmitting ultrasound waves at said treatment region using an ultrasound apparatus, wherein said ultrasound apparatus is adapted to be worn on said treatment region by said treated person undergoing a physical exercise; and
applying electrical stimulation to said treatment region substantially concurrently to the transmission of said ultrasound waves using an electrical stimulation apparatus, to stimulate intermittent contractions of the muscles at said treatment region,
wherein said treated person is undergoing a physical exercise substantially concurrently to the transmission of said ultrasound waves and to the application of said electrical stimulation,
whereby the combination of said transmission of said ultrasound waves, said application of said electrical stimulation, and said physical exercise, facilitates the release of triglycerides, fat molecules and toxins from stored fat within said treatment region, and accelerates the relative proportion of fat catabolism in said treatment region.

55. The method of claim 54, wherein said procedure of transmitting comprises at least one selected from the list consisting of:
transmitting ultrasound waves in a frequency between 1-3 MHz;
altering the frequency of said transmitted ultrasound waves according to a frequency variation pattern over the course of a treatment session; and
transmitting ultrasound waves with an intensity between 1.5-2.1 W/cm².

56. The method of claim 54, wherein said physical exercise is performed at an intensity level of approximately 60% of maximum heart rate.

57. The method of claim 54, wherein said procedure of applying electrical stimulation comprises at least one selected from the list consisting of:
interferential stimulation;
premodulated interferential stimulation;
biphasic interferential stimulation;
interferential isoplanar (4 poles) interferential stimulation;
interferential vectorial (4 poles) interferential stimulation; and
medium frequency interferential stimulation.
applying electrical stimulation with a current intensity between 5-90 mA;
applying electrical stimulation with a frequency between 5-150 Hz; and
altering the frequency of said electrical stimulation according to a frequency variation pattern.

58. The method of claim 54, further comprising the procedure of exerting pressure onto said treatment region substantially concurrently to the transmission of said ultrasound waves at said treatment region.

59. The method of claim 58, wherein said pressure is exerted by manually kneading a portion of said ultrasound apparatus against said treatment region.

60. The method of claim 54, further comprising the procedure of massaging said treatment region, where said massaging comprises at least one selected from the list consisting of:
massaging substantially concurrently to the transmission of said ultrasound waves at said treatment region;
massaging using said ultrasound apparatus;
massaging using a massaging device; and
massaging manually.

61. The method of claim 56, further comprising the procedure of applying gel onto said treatment region prior to the transmission of said ultrasound waves at said treatment region.

62. The method of claim 54, further comprising the procedures of:
acquiring initial measurements of said treated person prior to said procedure of transmitting ultrasound waves; and
acquiring final measurements of said treated person after said procedure of transmitting ultrasound waves.

63. The method of claim 54, further comprising the procedure of recording the treatment.
